SEQUENCE LISTING (4 sequences)

SEQ ID NO: 1 - DNA, 861 bp, Unknown
  Obtained from environmental sample
atggacgcactgggatattacaacggaaattgggggcctctggacgagatgactgtgccgatgaacgacaggggctgctactttggagacggcgtatacgacgctacctgcgccgttaacggagttatttttgccctggatgagcacattgacaggtttttcaacagcgcaaagctcctggaaataaatattagcttaacaaaagaggaattgaaaaaaactttaaatgaaatgtactccaaagtggataaaggagagtacctggtttattggcaggtgactcgcggaacaggccggcgaagccatgtatttccggcaggcccttcaaatctctggattataattaagcccaatcacatcgacaatctttatagaaaaatcaagctcattaccatggatgatacccgcttcctacactgcaacatcaagacccttaaccttatacccaatgtcattgcttcccagcgggcgctggaagcgggttgccacgaggcggtatttcaccggggcgaaacggtaaccgagtgcgctcacagcaatgtccatatcattaaaaacggcaggtttattacccatccggcggacaacctgatcctccggggtacagcccgcagtcatttattgcaggcctgtgtcaggcttaacattccggtagacgaacgggaattttccctttcggaattattcgacgcggacgaggtgcttgtgtccagcagcggcacactcggccttagcgccgaagaaatcgacggaaaaaaagcgggagggaaagcgcctgaactgctaaaaaaaatccaggatgaagtgctgagggaatttatcgaagcgacaggctacacgcctgagtggagcagggtatag

SEQ ID NO: 2 - protein, 286 aa, Unknown
  Obtained from environmental sample
MDALGYYNGNWGPLDEMTVPMNDRGCYFGDGVYDATCAVNGVIFALDEHIDRFFNSAKLLEINISLTKEELKKTLNEMYSKVDKGEYLVYWQVTRGTGRRSHVFPAGPSNLWIIIKPNHIDNLYRKIKLITMDDTRFLHCNIKTLNLIPNVIASQRALEAGCHEAVFHRGETVTECAHSNVHIIKNGRFITHPADNLILRGTARSHLLQACVRLNIPVDEREFSLSELFDADEVLVSSSGTLGLSAEEIDGKKAGGKAPELLKKIQDEVLREFIEATGYTPEWSRV

SEQ ID NO: 3 - DNA, 690 bp, Unknown
  Obtained from environmental sample
atgcctatcgttgttacgaagatcgaccgacccagcgcggcggacgtcgaaaggatcgccgcctatggtgtcgcgaccttgcatgaagcgcaaggacgaaccgggttgatggcgtccaatatgcgcccaatctatcgccctgcgcacattgccgggcccgcggtgacctgccttgtggcgcctggcgacaattggatgatccatgtcgccgtcgaacagtgccagccgggagatgtcctggtcgtggtaccgaccagcccctgcgaagacggctatttcggcgatctgctggcgacctcgctgcggtcgcgcggggtcaaaggtctgatcatcgaggccggcgtacgcgatatcgcgacattgaccgagatgaaattcccggtctggtccaaggcggtgttcgcgcaaggaacggtcaaggagaccatcgccagcgtcaatgtgcccctcgtctgcgcgggcgcccgcatcgtgccgggcgatctgatcgttgccgacgacgacggggtcgtcgtgattccaagacgttccgttccggcggtcctttccagcgccgaggcccgcgaagagaaggaagcccgcaaccgcgcccgcttcgaagctggcgagctgggcctcgacgtctacaacatgcgccagcgcctggccgacaagggcttgcgctatgtcgagcggctgcccgaggaatag

SEQ ID NO: 4 - protein, 229 aa, Unknown
  Obtained from environmental sample
MPIVVTKIDRPSAADVERIAAYGVATLHEAQGRTGLMASNMRPIYRPAHIAGPAVTCLVAPGDNWMIHVAVEQCQPGDVLVVVPTSPCEDGYFGDLLATSLRSRGVKGLIIEAGVRDIATLTEMKFPVWSKAVFAQGTVKETIASVNVPLVCAGARIVPGDLIVADDDGVVVIPRRSVPAVLSSAEAREEKEARNRARFEAGELGLDVYNMRQRLADKGLRYVERLPEE

(19) United States
(12) Patent Application Publication
(10) Pub. No.: US 2012/0270281 A1
(43) Pub. Date: Oct. 25, 2012

(54) BIOREACTOR PERFORMANCE IN THE PRODUCTION OF MONATIN

(75) Inventors: Erin Kathleen Marasco, Excelsior, MN (US); Maribeth Rasmussen, Crystal, MN (US); Christopher William Solheid, Minneapolis, MN (US); Trent H. Pemble, St. Bonifacius, MN (US); Michael A. Porter, Maple Grove, MN (US)

(73) Assignee: CARGILL, INCORPORATED, Wayzata, MN (US)

(21) Appl. No.: 13/520,027

(22) PCT Filed: Dec. 30, 2010
(86) PCT No.: PCT/US2010/062603

§ 371 Date: Jun. 29, 2012

Related U.S. Application Data
(60) Provisional application No. 61/335,035, filed on Dec. 30, 2009.

(51) Int. Cl. C12P 17/10

(52) U.S. Cl. 435/106

(57) ABSTRACT

Methods and systems for increasing the production of monatin in a multi-step equilibrium pathway are described. Tryptophan and pyruvate are added to a bioreactor to form a mixture comprising monatin and a plurality of intermediates via a multi-step equilibrium pathway. The methods and systems include operating the bioreactor such that a temperature of the mixture in the bioreactor is less than 25 degrees Celsius, resulting in an increased production of monatin. In some embodiments, the temperature of the mixture in the bioreactor is between about 5 degrees Celsius and about 23 degrees Celsius; in other embodiments, the temperature is between about 10 degrees Celsius and about 18 degrees Celsius.

### CROSS-REFERENCE TO RELATED APPLICATIONS

This application claims priority to U.S. Provisional application Ser. No. 61/335,035, filed 30 Dec. 2009, entitled IMPROVED BIOREACTOR PERFORMANCE IN THE PRODUCTION OF MONATIN, which is incorporated herein by reference in its entirety.

### REFERENCE TO A “SEQUENCE LISTING”

A Sequence Listing is being electronically filed concurrently with the electronic filing of this application and is herein incorporated by reference.

### FIELD

The present disclosure relates to a method and system for producing monatin in a multi-step equilibrium pathway. In particular, the present disclosure relates to a method and system for operating a bioreactor at a reduced temperature to increase the production of monatin.

### BACKGROUND

Monatin (2-hydroxy-2-(indol-3-ylmethyl)-4-aminoglutaric acid) is a naturally occurring, high intensity or high potency sweetener that was originally isolated from the plant Sclerochiton ilicifolius, found in the Transvaal Region of South Africa. Monatin has the chemical structure:

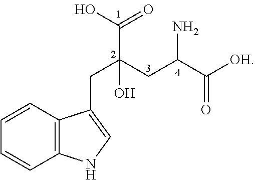

Because of various naming conventions, monatin is also known by a number of alternative chemical names, including: 2-hydroxy-2-(indol-3-ylmethyl)-4-aminoglutaric acid; 4-amino-2-hydroxy-2-(1H-indol -3 - ylmethyl)-pentanedioic acid; 4-hydroxy-4-(3-indolylmethyl)glutamic acid; and 3-(1-amino-1,3 -dicarboxy-3-hydroxy-but-4-yl)indole.

Monatin has two chiral centers thus leading to four potential stereoisomeric configurations: the R,R configuration (the “R,R stereoisomer” or “R,R monatin”); the S,S configuration (the “S,S stereoisomer” or “S,S monatin”); the R,S configuration (the “R,S stereoisomer” or “R,S monatin”); and the S,R configuration (the “S,R stereoisomer” or “S,R monatin”).

Reference is made to WO 2003/091396 A2, which discloses, inter alia, polypeptides, pathways, and microorganisms for in vivo and in vitro production of monatin. WO 2003/091396 A2 (see, e.g., FIGS. 1-3 and 11-13) and U.S. Patent Publication No. 2005/282260 describe the production of monatin from tryptophan through multi-step pathways involving biological conversions with polypeptides (proteins) or enzymes. One pathway described involves converting tryptophan to indole-3-pyruvate (“I3P”) (reaction (1)), converting indole-3-pyruvate to 2-hydroxy 2-(indol-3-ylmethyl)-4-keto glutaric acid (monatin precursor, “MP”) (reaction (2)), and converting MP to monatin (reaction (3)). The three reactions can be performed biologically, for example, with enzymes.

### SUMMARY

Provided herein are methods and systems for improved performance of a bioreactor used in the production of monatin. Monatin may be produced biosynthetically via a multi-step equilibrium pathway that includes the enzymatic conversion of tryptophan to indole-3-pyruvate (I3P), I3P to 2-hydroxy 2-(indol-3-ylmethyl)-4-keto glutaric acid (MP), and MP to monatin. Tryptophan and pyruvate are added to a bioreactor to form a mixture of monatin and intermediates. An increased production of monatin results from operating the bioreactor such that a temperature of the mixture is less than 25 degrees Celsius.

In one embodiment, a method of making monatin in a multi-step equilibrium pathway includes adding tryptophan and pyruvate to a reactor to form a mixture comprising monatin and a plurality of intermediates via a multi-step equilibrium pathway, and adding at least one enzyme to the reactor to facilitate at least one reaction in the multi-step equilibrium pathway. The method includes operating the reactor under conditions such that a temperature of the mixture in the reactor is between about 5 degrees Celsius and about 23 degrees Celsius. In some aspects, the temperature is between about 10 and about 18 degrees Celsius. In some aspects, the temperature is between about 12 and about 16 degrees Celsius.

In another embodiment, a method of producing monatin includes adding tryptophan and pyruvate to a reactor to produce a mixture of monatin and intermediates via a multi-step equilibrium pathway in which tryptophan is converted to indole-3-pyruvate, indole-3-pyruvate is converted to 2-hydroxy 2-(indol-3-ylmethyl)-4-keto glutaric (MP), and MP is converted to monatin. The method further includes adding an aminotransferase to the reactor to catalyze the conversion of tryptophan to indole-3-pyruvate and the conversion of MP to monatin, and adding an aldolase to the reactor to catalyze the conversion of indole-3-pyruvate to MP. The temperature of the mixture is maintained between about 5 and about 23 degrees Celsius. In some aspects, the temperature of the mixture is maintained between about 10 and about 18 degrees Celsius. In some aspects, the temperature is maintained at about 15 degrees Celsius. In some aspects, the mixture is removed from the reactor after the multi-step equilibrium pathway reaches equilibrium. In some aspects, the pH of the mixture is maintained between about 7 and about 9; and in other aspects, maintaining the pH of the mixture includes adding a hydroxide to the reactor. The hydroxide may include at least one of sodium hydroxide and potassium hydroxide. In some aspects, the monatin in the mixture is a stereoisomecially-enriched R,R monatin. In some aspects, an amount of tryptophan added to the reactor is such that a concentration of tryptophan in the mixture is greater than a solubility limit of tryptophan in the mixture.

In another embodiment, a method of producing a stereoisomeric ally-enriched R,R monatin includes adding D-tryptophan and pyruvate to a reactor to produce a mixture of stereoisomerically-enriched R,R monatin and intermediates, wherein the tryptophan is converted to indole-3-pyruvate, indole-3-pyruvate to 2-hydroxy 2-(indol-3-ylmethyl)-4-keto glutaric (MP), and MP to monatin. The method further includes adding a D-aminotransferase and an R-specific aldolase to the reactor, wherein the D-aminotransferase catalyzes at least one of the conversion of D-tryptophan to indole-3-pyruvate and R-MP to R,R-monatin, and the R-specific aldolase catalyzes the conversion of indole-3-pyruvate to R-MP. At least one additive is added to the reactor to stabilize at least one of the intermediates, the D-aminotransferase and the R-specific aldolase. The temperature of the mixture is maintained between about 5 degrees Celsius and about 23 degrees Celsius. In some aspects, the temperature is maintained between about 10 and about 18 degrees Celsius. In some aspects, the temperature is maintained between about 12 and about 16 degrees Celsius; in other aspects, the temperature is maintained at about 15 degrees Celsius. In some aspects, the additive added to the reactor is an enzymatic cofactor. The additive may include at least one of potassium phosphate, sodium phosphate, magnesium chloride, pyridoxal phosphate, and a surfactant. In some aspects, the method may further include removing the mixture from the reactor after at least about 8 hours; in other aspects, the mixture is removed after at least about 24 hours.

In another embodiment, a method of producing monatin includes adding tryptophan and pyruvate to a reactor to produce a mixture of monatin and intermediates via a multi-step equilibrium pathway in which tryptophan is converted to indole-3-pyruvate, indole-3-pyruvate is converted to 2-hydroxy 2-(indol-3-ylmethyl)-4-keto glutaric (MP), and MP is converted to monatin. The method further includes adding an aminotransferase to the reactor to catalyze the conversion of tryptophan to indole-3-pyruvate and the conversion of MP to monatin, and adding an aldolase to the reactor to catalyze the conversion of indole-3-pyruvate to MP. The temperature of the mixture is maintained at a first temperature for a predetermined time, with the first temperature being less than or equal to about 25 degrees Celsius. After the predetermined time, the temperature of the mixture is maintained at a second temperature that is less than the first temperature. In some aspects, the second temperature is between about 10 and about 18 degrees Celsius. In some aspects, the predetermined time is less than 8 hours.

In yet another embodiment, a method of making monatin includes adding tryptophan and pyruvate to a reactor to form a mixture comprising monatin and a plurality of intermediates via a multi-step equilibrium pathway, adding at least one enzyme to the reactor to facilitate at least one reaction in the multi-step equilibrium pathway, and operating the bioreactor such that a temperature of the mixture is less than or equal to about 25 degrees Celsius for a predetermined time. After the predetermined time, the temperature of the mixture is decreased as a function of reaction time, until a minimum temperature is reached. In some aspects, the minimum temperature is equal to about 13 degrees Celsius. In some aspects, the minimum temperature is equal to about 10 degrees Celsius. In some aspects, the multi-step equilibrium pathway includes a conversion of tryptophan to indole-3-pyruvate, indole-3-pyruvate to 2-hydroxy 2-(indol-3-ylmethyl)-4-keto glutaric (MP), and MP to monatin. In some aspects, the at least one enzyme added to the reactor may include an aminotransferase, a racemase, and an aldolase.

In some aspects, the monatin produced is a stereoisomerically-enriched R,R monatin. In some aspects, the mixture in the bioreactor is maintained at a pH between about 7 and about 9. In some aspects, an amount of tryptophan added to the reactor is such that a concentration of tryptophan in the mixture is greater than a solubility limit of tryptophan in the mixture.

The details of one or more non-limiting embodiments of the invention are set forth in the description below. Other embodiments of the invention should be apparent to those of ordinary skill in the art after consideration of the present disclosure.

### BRIEF DESCRIPTION OF THE DRAWINGS

FIG. 1 is a block diagram of an exemplary system for the production of monatin.

FIG. 2 is a block diagram of another system for the production of monatin.

### DETAILED DESCRIPTION

Monatin has an excellent sweetness quality, and depending on a particular composition, monatin may be several hundred times sweeter than sucrose, and in some cases thousands of times sweeter than sucrose. As stated above, monatin has four stereoisomeric configurations. The S,S stereoisomer of monatin is about 50-200 times sweeter than sucrose by weight. The R,R stereoisomer of monatin is about 2000-2400 times sweeter than sucrose by weight. As used herein, unless otherwise indicated, the term “monatin” is used to refer to compositions including any combination of the four stereoisomers of monatin (or any of the salts thereof), including a single isomeric form.

Monatin may be synthesized in whole or in part by one or more of a biosynthetic pathway, chemically synthesized, or isolated from a natural source. If a biosynthetic pathway is used, it may be carried out in vitro or in vivo and may include one or more reactions such as the equilibrium reactions provided below as reactions (1)-(3). In one embodiment, this is a biosynthetic production of monatin via enzymatic conversions starting from tryptophan and pyruvate and following the three equilibrium reactions below:

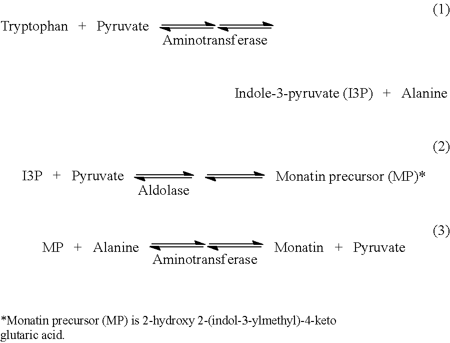

The following two side-reactions may also occur, which results in production of hydroxymethyl-oxo-glutarate (HMO) and hydroxymethylglutamate (HMG):

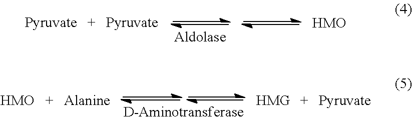

In the pathway shown above, in reaction (1), tryptophan and pyruvate are enzymatically converted to indole-3-pyruvate (I3P) and alanine in a reversible reaction. As exemplified above, an enzyme, here an aminotransferase, is used to facilitate (catalyze) this reaction. In reaction (1), tryptophan donates its amino group to pyruvate and becomes I3P. In reaction (1), the amino group acceptor is pyruvate, which then becomes alanine as a result of the action of the aminotransferase. The amino group acceptor for reaction (1) is pyruvate; the amino group donor for reaction (3) is alanine. The formation of indole-3-pyruvate in reaction (1) can also be performed by an enzyme that utilizes other α-keto acids as amino group acceptors, such as oxaloacetic acid and α-keto-glutaric acid. Similarly, the formation of monatin from MP (reaction (3)) can be performed by an enzyme that utilizes amino acids other than alanine as the amino group donor. These include, but are not limited to, aspartic acid, glutamic acid, and tryptophan.

Some of the enzymes useful in connection with reaction (1) may also be useful in connection with reaction (3). For example, aminotransferase may be useful for both reactions (1) and (3). The equilibrium for reaction (2), the aldolase-mediated reaction of indole-3-pyruvate to form MP (i.e. the aldolase reaction), favors the cleavage reaction generating indole-3-pyruvate and pyruvate rather than the addition reaction that produces the alpha-keto acid precursor to monatin (i.e. MP). The equilibrium constants of the aminotransferase-mediated reactions of tryptophan to form indole-3-pyruvate (reaction (1)) and of MP to form monatin (reaction (3)) are each thought to be approximately one. Methods may be used to drive reaction (3) from left to right and prevent or minimize the reverse reaction. For example, an increased concentration of alanine in the reaction mixture may help drive forward reaction (3). Reference is made to US Publication No. 2009/0198072 (application Ser. No. 12/315,685), which is also assigned to Cargill, the assignee of this application.

The overall production of monatin from tryptophan and pyruvate is referred to herein as a multi-step pathway or a multi-step equilibrium pathway. A multi-step pathway refers to a series of reactions that are linked to each other such that subsequent reactions utilize at least one product of an earlier reaction. In such a pathway, the substrate (for example, tryptophan) of the first reaction is converted into one or more products, and at least one of those products (for example, indole-3-pyruvate) can be utilized as a substrate for the second reaction. The three reactions above are equilibrium reactions such that the reactions are reversible. As used herein, a multi-step equilibrium pathway is a multi-step pathway in which at least one of the reactions in the pathway is an equilibrium or reversible reaction.

Reactions (1)-(3) are commonly performed at a temperature of approximately 25 degrees Celsius, since this was believed to be the optimum temperature for enzymatic activity. However, the inventors unexpectedly observed a higher concentration of monatin produced when the reactions were maintained at a lower temperature. For example, up to a 32 percent increase in monatin concentration was observed by reducing the temperature of the reactions in the bioreactor from 25 degrees Celsius to 13 degrees Celsius (see Example 7 below). In one aspect, this was surprising because enzymes typically have faster rates at higher temperatures. The present disclosure focuses on a method and system for increasing the production of monatin in a reactor by maintaining the monatin producing reactions at a temperature less than 25 degrees Celsius. In some embodiments, the method and system includes maintaining the reactions at essentially a constant temperature throughout the operation of the reactor. In some embodiments, the method and system includes reducing the temperature after a predetermined time of operating the reactor.

Because the R,R stereoisomer of monatin is the sweetest of the four stereoisomers, it may be preferable to selectively produce R,R monatin. For purposes of this disclosure, the focus is on the production of R,R monatin. However, it is recognized that the present disclosure is applicable to the production of any of the stereoisomeric forms of monatin (R,R; S,S; S,R; and R,S), alone or in combination.

In some embodiments, the monatin consists essentially of one stereoisomer—for example, consists essentially of S,S monatin or consists essentially of R,R monatin. In other embodiments, the monatin is predominately one stereoisomer—for example, predominately S,S monatin or predominately R,R monatin. “Predominantly” means that of the monatin stereoisomers present in the monatin, the monatin contains greater than 90% of a particular stereoisomer. In some embodiments, the monatin is substantially free of one stereoisomer—for example, substantially free of S,S monatin. “Substantially free” means that of the monatin stereoisomers present in the monatin, the monatin contains less than 2% of a particular stereoisomer. In some embodiments, the monatin is a stereoisomerically-enriched monatin mixture. “Stereoisomerically-enriched monatin mixture” means that the monatin contains more than one stereoisomer and at least 60% of the monatin stereoisomers in the mixture is a particular stereoisomer. In other embodiments, the monatin contains greater than 60%, 65%, 70%, 75%, 80%, 85%, 90%, 95%, 98% or 99% of a particular monatin stereoisomer. In another embodiment, a monatin composition comprises a stereoisomerically-enriched R,R-monatin, which means that the monatin comprises at least 60% R,R monatin. In other embodiments, stereoisomerically-enriched R,R-monatin comprises greater than 60%, 65%, 70%, 75%, 80%, 85%, 90%, 95%, 98% or 99% of R,R monatin.

For example, to produce R,R monatin using the three-step pathway shown above (reactions (1)-(3)), the starting material may be D-tryptophan, and the enzymes may be a D-aminotranferase and an R-specific aldolase. The three reactions, which are shown below, may be carried out in a single reactor or a multiple-reactor system.

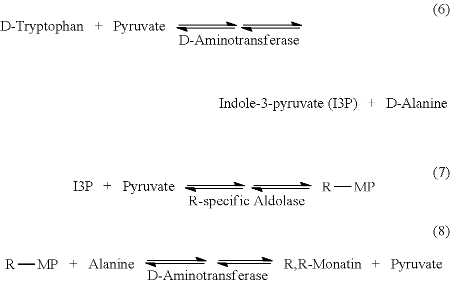

In an embodiment in which a single reactor is used, the two enzymes (i.e. the D-aminotransferase and the R-specific aldolase) may be added at the same time and the three reactions may run simultaneously. The same enzyme may be used to catalyze reactions (6) and (8). A D-aminotransferase is an enzyme with aminotransferase activity that selectively produces, in the reactions shown above, D-alanine and R,R-monatin. An R-specific aldolase is an enzyme with aldolase activity that selectively produces R-MP, as shown in reaction (7) above. Although a focus in the present disclosure is on the production of R,R monatin using a single reactor via the reactions shown immediately above, it is recognized that the method and system of maintaining the monatin producing reactions at a lower temperature is applicable to the production of any of the stereoisomeric forms of monatin, and to the production of R,R monatin using an alternative pathway.

There are multiple alternatives to the above pathway (i.e. reactions (6)-(8)) for producing R,R-monatin. For example, L-tryptophan may be used as a starting material instead of D-tryptophan. In that case, an L-aminotransferase may be used to produce indole-3-pyruvate and L-alanine from L-tryptophan. Because L-alanine is produced, this pathway may require the use of an alanine racemase to convert the L-alanine to D-alanine, thus adding a fourth reaction to the monatin production pathway. (D-alanine is required to produce R,R monatin from the R- stereoisomer of monatin precursor (R-MP). In addition to requiring another enzyme (alanine racemase), undesired side reactions may also occur in this pathway. For example, L-alanine may react with the L-aminotransferase to produce R,S-monatin, or D-alanine may react with I3P to form D-tryptophan, resulting in a racemate of L-tryptophan and D-tryptophan, which has poor solubility. Some disadvantages of this pathway may be avoided by using a two reactor system as opposed to a single reactor system. It is recognized that there are additional alternatives not specifically disclosed herein for performing the three-step equilibrium pathway to produce monatin. The method and system described herein for maintaining the monatin producing reactions at a lower temperature is applicable to alternative pathways for producing monatin.

As described above, in some pathways, it may be preferable to perform the monatin producing reactions in two or more separate reactors, while in other pathways it may be preferable to use a single reactor system. The decision to use a one reactor or a multiple reactor system may depend, in part, on whether D-tryptophan or L-tryptophan is used as a starting material. A single reactor system is obviously simpler in design, eliminating the need for a second reactor, as well as eliminating, in some cases, a need for a separation step between the first and second reactors. It is recognized that the method and system described herein for maintaining the monatin producing reactions at a lower temperature is applicable regardless of whether the system has a single reactor or multiple reactors.

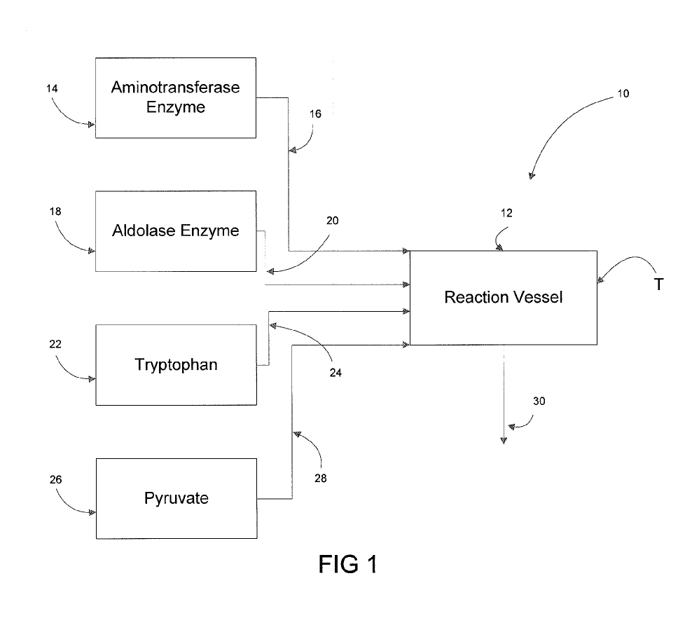

FIG. 1 is a block diagram of exemplary system 10 for producing monatin, which includes reaction vessel 12, also referred to herein as a bioreactor. The inputs to reaction vessel 12 include aminotransferase enzyme 14 (conveyable to vessel 12 through conduit 16), aldolase enzyme 18 (conveyable to vessel 12 through conduit 20), and starting materials tryptophan 22 and pyruvate 26, conveyable to vessel 12 through conduits 24 and 28, respectively Aminotransferase 14 and aldolase 18 catalyze reactions (6)-(8) shown above to produce monatin from tryptophan 22. The resulting reaction mixture inside reaction vessel 12 may be removed through conduit 30 after a given period of time, which may be, for example, at a time about equal to or greater than a time for the reactions to reach equilibrium. In some embodiments, the time to reach equilibrium may be about 24 hours. In other embodiments, the time to reach equilibrium may be greater or less than about 24 hours. In other embodiments, the reaction mixture may be removed at a time that is less than a time for the reactions to reach equilibrium. The reaction mixture may comprise monatin, and one or more of MP, I3P, alanine, tryptophan, pyruvate, HMG and HMO. It is recognized that additional reactants or components not shown in FIG. 1 may be added to reaction vessel 12 to aid in the production of monatin. A temperature T of the reaction mixture inside vessel 12 may be measured using a thermocouple or other temperature measuring device.

A primary goal of system 10 is to maximize the amount of monatin produced in reaction vessel 12. An increase in monatin is observed when temperature T of the reaction mixture inside vessel 12 is below 25 degrees Celsius. Thus, system 10 includes a method and/or device for controlling temperature T of the mixture inside reaction vessel 12. In some embodiments, vessel 12 includes a heating and cooling loop (not shown) that maintains the temperature of the mixture inside vessel 12 at a set point temperature. It is recognized that temperature T of the mixture inside vessel 12 may be controlled by various methods known to one of skill in the art.

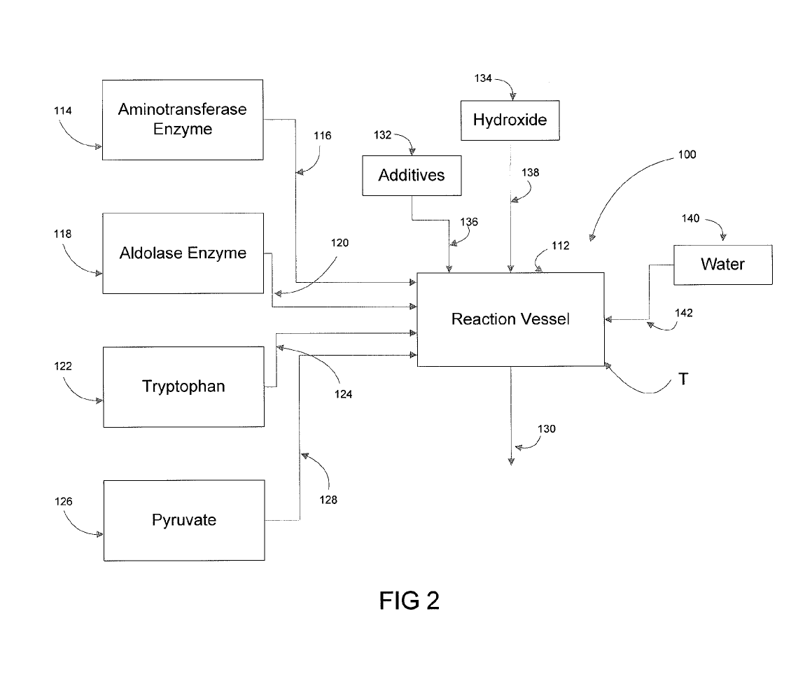

FIG. 2 illustrates an alternative embodiment to the system of FIG. 1. FIG. 2 is a block diagram of system 100 for the production of monatin, which includes the same components as FIG. 1, but also includes additives 132 and hydroxide 134. Additives 132 (conveyable to vessel 112 through conduit 136) may include components for improving a performance of either or both of enzymes 114 and 118. Additives 132 may include components for improving the stability of either or both of enzymes 114 and 118 or any of the starting materials or intermediates in the reaction pathway. Specific examples of additives 132 are described below. Hydroxide 134 (conveyable to vessel 112 through conduit 138) is used to adjust or maintain a pH of the reaction mixture inside vessel 112, and is discussed further below. System 100 may also include water 140, conveyable to vessel 112 through conduit 142. System 100 may include additional components not shown in FIG. 2. For example, an additional input to reactor vessel 112 may be alanine, which is used, in addition to the alanine formed in reaction (6), to drive forward the reaction of MP to monatin.

In some embodiments, a pH of the reaction mixture inside vessel 112 is between about 7 and about 9. In other embodiments, the pH is between about 7.5 and about 8.2.

At least some of the intermediates formed in the production of monatin (for example, I3P) are unstable in the presence of oxygen. As such, in some embodiments, system 100 operates in an oxygen-free environment. Before starting system 100, reactor 112 is purged with an inert gas, for instance nitrogen. While system 100 is running, reactor 112 is kept under a nitrogen overlay. Water 140 added to reactor 112 is degassed and sparged with nitrogen for oxygen removal. Reactor 112 may operate at a positive nitrogen pressure, for example, with a sweep of 0.400 cfm.

In some embodiments, system 100 is operated by performing the following steps in the designated order. It is recognized that the method and system of operating the bioreactor at a reduced temperature is applicable to monatin producing systems that deviate from the steps or the sequence of steps provided herein. Before beginning to operate system 100, reactor 112 may be purged with nitrogen for approximately 15 minutes prior to the addition of any of the inputs to reactor 112. Tryptophan 122 and pyruvate 126, both in solid form, may be added to reactor 112, along with degassed water 140. Reactor 112 may include an agitator (not shown), which is used to dissolve at least a portion of tryptophan 122 and pyruvate 126 in water 140. Next, additives 132 may be conveyed to reactor 112. In some embodiments, additives 132 include a salt form of the phosphate anion, including, but not limited to, sodium phosphate and potassium phosphate; a salt form of the magnesium cation, including, but not limited to, magnesium chloride; a composition to deliver the active form of vitamin B₆ (pyridoxal-5-phosphate; PLP); and a surfactant. Hydroxide 134 may be added to reactor 112 in order to adjust and/or maintain a pH of the mixture. In some embodiments, hydroxide 134 is added to reactor 112 prior to addition of enzymes 114 and 118, and a pH probe may be used to measure pH before proceeding. Finally, enzymes 114 and 118 may be conveyed to reactor 112.

Reactor 112 includes a temperature sensing device such as a thermocouple for measuring temperature T of the reaction mixture inside vessel 112. In some embodiments, temperature T is maintained at essentially the same temperature throughout operation of vessel 112. In other embodiments, temperature T is maintained within a given temperature range. It is recognized by one of skill in the art that temperature fluctuations will occur within an acceptable margin of error for a given temperature or a given temperature range. To maximize an amount of monatin produced in reactor 112, temperature T is maintained at or below about 25 degrees Celsius. In some embodiments, temperature T is maintained between about 5 and about 23 degrees Celsius. In other embodiments, temperature T is maintained between about 10 and about 18 degrees Celsius; in yet other embodiments, between about 12 and about 16 degrees Celsius. It is recognized that temperature T may be maintained at lower temperatures, for example, between about 0 and about 5 degrees Celsius. As described above, temperature T may be adjusted and maintained using any method known to one of skill in the art, including manual or automated temperature control. Temperature T is adjusted until it matches the set point temperature.

The set point temperature, and hence temperature T of the reaction mixture, may be based, in part, on how long the reactions are intended to run in reactor 112. In some embodiments, the run time may be about 24 hours. In other embodiments, the run time may be about 8 hours; in other embodiments, about 48 hours; and in yet other embodiments, about 72 hours. As recognized by one of skill in the art, an increase in reaction time, due to a reduction in operating temperature, may be offset by adjusting other parameters in the operation of systems 10 and 100. Although high amounts of monatin may be produced at temperatures of 15 degrees Celsius and lower, a determination of temperature T may depend, in part, on the costs of running the reaction for a longer period of time and/or the cooling costs for adjusting temperature T.

In some embodiments, the set point temperature of the reaction mixture may change after the reaction has been running for a certain period of time. A first temperature may be used as the set point temperature at the start of the reaction and up until a predetermined time; then a second temperature may be used as the set point temperature for the remainder of the run time. As shown in the examples below, the initial rate of monatin production generally increases as a function of an increase in the temperature of the reaction mixture. However, after longer reaction times, the difference in monatin production as a function of temperature is significantly reduced. Moreover, the examples show that the lower operating temperatures eventually overtake the higher operating temperatures, in terms of producing more monatin. As an example, temperature T of the reaction mixture may initially be equal to about 23 degrees Celsius; after running for approximately 6 hours, temperature T may be reduced to about 15 degrees Celsius.

In other embodiments, more than two different operating temperatures may be used. In yet another embodiment, temperature T of the reaction mixture may gradually decrease as a function of the run time. For example, temperature T may start out at 25 degrees Celsius at the beginning of the run, and after a given time period (for example, 4 hours) temperature T may be reduced by 2 degrees every four hours, until a minimum set point temperature (for example, 13 degrees Celsius) is reached.

In some embodiments, more than one bioreactor may be used for the production of monatin and the reaction mixture in each of the bioreactors may be maintained at a different temperature. For example, a first reactor may be used to carry out the monatin-producing reactions for a given period of time at a first temperature. The reaction mixture may then be transferred to a second reactor in which the reaction mixture is maintained at a second temperature that is lower than the first temperature.

In some embodiments, an amount of tryptophan added to reactors 12 and 112 of systems 10 and 100, respectively, is higher than the solubility limit of tryptophan in the reaction mixture, in which case some of the tryptophan in the mixture would not be dissolved. It is recognized that the solubility limit of tryptophan in the mixture depends in part on the temperature of the reaction mixture (i.e. the solubility limit decreases as the reaction temperature decreases).

Aspects of the invention are illustrated in the following non-limiting examples.

### EXAMPLES

### Example 1

Derivatization of Monatin Intermediates (Indole-3-Pyruvic Acid, Hydroxymethyloxyglutaric Acid (HMO), Monatin Precursor, and Pyruvate) with O-(4-Nitrobenzyl)hydroxylamine hydrochloride (NBHA)

In the process of monatin production various intermediate compounds are formed and utilized. These compounds include: indole-3-pyruvic acid, hydroxymethyloxyglutaric acid (HMO), monatin precursor, and pyruvate. The ketone functional group on these compounds can be derivatized with O-(4-Nitrobenzyl)hydroxylamine hydrochloride (NBHA) to form a stable compound for analysis.

UPLC/UV Analysis of Monatin Intermediates (Indole-3-Pyruvic Acid, Hydroxymethyloxyglutaric Acid, Monatin Precursor, and Pyruvate)

A Waters Acquity UPLC instrument including a Waters Acquity Photo-Diode Array (PDA) absorbance monitor is used for the analysis of the intermediate compounds. UPLC separations were made using a Waters Acquity HSS T3 1.8mm 1×150 mm column at 50° C. The UPLC mobile phase consisted of A) water containing 0.3% formic acid and 10 mM ammonium formate and B) 50/50 acetonitrile/methanol containing 0.3% formic acid and 10 mM ammonium formate.

The gradient elution was linear from 5% B to 40% B, 0-1.5 min, linear from 40% B, to 50% B, 1.5-4.5 min, linear from 50% B to 90% B, 4.5-7.5 min, linear from 90% B to 95% B, 7.5-10.5 min, with a 3 min re-equilibration period between runs. The flow rate was 0.15 mL/min from 0-7.5 mM, 0.18mL/min from 7.5-10.5 min, 0.19 mL/min from 10.5-11 min, and 0.15 mL/min from 11-13.5 min. PDA absorbance was monitored at 270 nm.

Sample concentrations are calculated from a linear least squares calibration of peak area at 270nm to known concentration, with a minimum coefficient of determination of 99.9%.

### Example 2

UPLC/UV Analysis of monatin and tryptophan

Analyses of mixtures for monatin and tryptophan derived from biochemical reactions were performed using a Waters Acquity UPLC instrument including a Waters Acquity Photo-Diode Array (PDA) absorbance monitor. UPLC separations were made using an Agilent XDB C8 1.8 μm 2.1×100 mm column (part # 928700-906) at 30° C. The UPLC mobile phase consisted of A) water containing 0.1% formic B) acetonitrile containing 0.1% formic acid.

The gradient elution was linear from 5% B to 40% B, 0-4 min, linear from 40% B, to 90% B, 4-4.2 min, isocratic from 90% B to 90% B, 4.2-5.2 min, linear from 90% B to 5% B, 5.2-5.3 min, with a 1.2 min re-equilibration period between runs. The flow rate was 0.5 mL/min, and PDA absorbance was monitored at 280 nm

Sample concentrations are calculated from a linear least squares calibration of peak area at 280 nm to known concentration, with a minimum coefficient of determination of 99.9%.

### Example 3

Liquid Chromatography-Post Column Derivatization with OPA, Fluorescence Detection of Amino Acids, including: Hydroxymethyl glutamate (HMG) and Alanine

Analyses of mixtures for HMG and alanine derived from biochemical reactions were performed using a Waters Alliance 2695 and a Waters 600 configured instrument with a Waters 2487 Dual Wavelengths Absorbance Detector and Waters 2475 Fluorescence Detector as a detection system. HPLC separations were made using both a Phenomenex Aqua C18 125A, 150 mm×2.1 mm, 3μ, cat #00F4311B0, and a Phenomenex Aqua C18 125A, 30 mm×2.1 mm, 3μ, cat # 00A4311B0 at 55° C. The HPLC mobile phase consisted of A) 0.6% acetic acid with 1% MeOH.

The flow rate was (100% A) 0.2 mL/min from 0-3.5 min, 0.24 mL/min from 3.5-6.5 min, 0.26 mL/min from 6.5-10.4 min, and 0.2 mL/min from 10.4-11 min. Absorbance was monitored at 336 nm. Sample concentrations are calculated from a linear least squares calibration of peak area at 336nm to known concentration, with a minimum coefficient of determination of 99.9%.

### Example 4

Chiral LC/MS/MS (MRM) Measurement of Monatin

Determination of the stereoisomer distribution of monatin in biochemical reactions was accomplished by derivatization with 1-fluoro-2-4-dinitrophenyl-5-L-alanine amide 30 (FDAA), followed by reversed-phase LC/MS/MS MRM measurement.

LC/MS/MS Multiple Reaction Monitoring for the Determination of the Stereoisomer Distribution of Monatin

Analyses were performed using the Waters/Micromass® liquid chromatography-tandem mass spectrometry (LC/MS/MS) instrument including a Waters 2795 liquid chromatograph with a Waters 996 Photo-Diode Array (PDA) absorbance monitor placed in series between the chromatograph and a Micromass® Quattro Ultima® triple quadrupole mass spectrometer. The LC separations capable of separating all four stereoisomers of monatin (specifically FDAA-monatin) were performed on a Phenomenex Luna® 2.0×250 mm (3 μm) C18 reversed phase chromatography column at 40° C. The LC mobile phase consisted of A) water containing 0.05% (mass/volume) ammonium acetate and B) Acetonitrile. The elution was isocratic at 13% B, 0-2 min, linear from 13% B to 30% B, 2-15 min, linear from 30% B to 80% B, 15-16 min, isocratic at 80% B 16-21 min, and linear from 80% B to 13% B, 21-22 min, with a 8 min re-equilibration period between runs. The flow rate was 0.23 mL/min, and PDA absorbance was monitored from 200 nm to 400 nm. All parameters of the ESI-MS were optimized and selected based on generation of deprotonated molecular ions ([M-H]-) of FDAA-monatin, and production of characteristic fragment ions. The following instrumental parameters were used for LC/MS analysis of monatin in the negative ion ESI/MS mode: Capillary: 3.0 kV; Cone: 40 V; Hex 1: 15 V; Aperture: 0.1 V; Hex 2: 0.1 V; Source temperature: 120° C.; Desolvation temperature: 350° C.; Desolvation gas: 662 L/h; Cone gas: 42 L/h; Low mass resolution (Q1): 14.0; High mass resolution (Q1): 15.0; Ion energy: 0.5; Entrance: 0 V; Collision Energy: 20; Exit: 0 V; Low mass resolution (Q2): 15; High mass resolution (Q2): 14; Ion energy (Q2): 2.0; Multiplier: 650. Three FDAA-monatin-specific parent-to-daughter transitions were used to specifically detect FDAA-monatin. Identification of FDAA-monatin stereoisomers was based on chromatographic retention time as compared to purified monatin stereoisomers.

### Example 5

Derivatization of Amino Acids with 9-fluorenylmethyl chloroformate (FMOC-chloride or FMOC-Cl)

These amino acids include: Monatin, Alanine, Hydroxymethyl glutamate (HMG), and Tryptophan. The amine functional group on these compounds can be derivatized with 9-fluorenylmethyl to form a stable compound for analysis.

UPLC/UV Analysis of Monatin Amino Acids (Monatin, Alanine, Hydroxymethyl glutamate (HMG), and Tryptophan

A Waters Acquity UPLC instrument including a Waters Acquity Photo-Diode Array (PDA) absorbance monitor is used for the analysis of the intermediate compounds. UPLC separations were made using a Waters Acquity HSS T3, 100 mm×2.1 mm×1.8 μm, (part #186003539) at 45° C. The UPLC mobile phase consisted of A) water containing 0.2% formic acid B) acetonitrile.

The gradient elution was linear from 10% B to 30% B, 0-1.0 min, linear from 30% B, to 37% B, 1.0-2.5 min, curved 7 from 37% B to 64% B, 2.5-5.7 min, curved 5 from 64% B to 90% B, 5.7-7.5 min, linear from 90% B to 95% B, 7.5-8.0 min, linear from 95% B to 10% B, 8.0-8.1 min, with a 1.4 min re-equilibration period between runs. The flow rate was 0.6 mL/min PDA absorbance was monitored at 265nm.

Sample concentrations are calculated from a linear least squares calibration of peak area at 265nm to known derivatized external standard, with a minimum coefficient of determination of 99.9%.

### Example 6

The present example evaluated the production of monatin in a single bioreactor over time, as a function of different operating temperatures ranging between 13 and 32 degrees Celsius. The three step pathway for the production of monatin (see reactions (6)-(8) above) was carried out at 300 mL final volume in 0.7 L INFORS (Infors AG, Bottmingen, Switzerland) bioreactors. Each bioreactor was configured for automated control of agitation, temperature and pH.

Solutions containing 130 mM D-tryptophan, 200 mM sodium pyruvate, 10 mM potassium phosphate (pH 7.8), 1 mM MgCl₂, 0.01% (v/v) Tween 80 and 0.05 mM pyridoxal-5-phosphate (PLP) were prepared in the bioreactors. D-tryptophan and sodium pyruvate were added as solids, the remaining components were added from degassed stock solutions.

The temperature of the mixture inside each reactor was maintained throughout operation at the predetermined temperatures of 13, 18, 22, 28 and 32 degrees Celsius. The pH was adjusted and maintained at a pH of 7.8 by adding sodium hydroxide to the bioreactor. A D-aminotransferase (see SEQ ID NO:1 and SEQ ID NO:2) and an aldolase (see SEQ ID NO:3 and SEQ ID NO:4) were added as clarified cell extracts to a final concentration of 0.2 and 0.02 g/L, respectively. The mixture inside the reactor was agitated at 250 rpm and maintained at the controlled temperature under a nitrogen headspace.

The progress of the reaction was followed by measuring pyruvate, indole-3-pyruvate, HMO, monatin precursor (MP) using the analytical method of Example 1. Tryptophan and monatin were measured using the analytical method of Example 2, and alanine and HMG were measured using the analytical method of Example 3.

The concentration of monatin at various reaction times is shown in Table 1 below.

TABLE 1
Monatin formation (mM) over time at 13° C., 18° C., 22° C.,
28° C. and 32° C.

| | 13° C. | 18° C. | 22° C. | 28° C. | 32° C. |
|---|---|---|---|---|---|
| 0 hr | 0.3 | 0.3 | 0.2 | 0.2 | 0.2 |
| 0.25 hr | 0.3 | 0.2 | 0.3 | 0.3 | 0.4 |
| 0.5 hr | 0.3 | 0.4 | 0.5 | 0.8 | 1.1 |
| 0.75 hr | 0.4 | 0.6 | 0.9 | 1.4 | 1.7 |
| 1 hr | 0.6 | 1.0 | 1.4 | 2.1 | 2.2 |
| 2 hr | 1.8 | 3.0 | 3.7 | 4.7 | 3.8 |
| 4 hr | 4.4 | 6.6 | 7.1 | 7.8 | 4.7 |
| 24 hr | 21.4 | 22.3 | 17.1 | 14.5 | 6.9 |

The results in Table 1 illustrate that after approximately four hours, more monatin is produced when the reaction mixture is maintained at 22° C. and 28° C., as compared to 13° C., 18° C. and 32° C. However, after the reaction has been running for about 24 hours, significantly more monatin is produced at 13° C. and 18° C. At 13° C., 25% more monatin is produced at 24 hours as compared to 22° C. At 18° C., 30% more monatin is produced at 24 hours as compared to 22° C.

### Example 7

Based on the results from Example 6 above, the present example was used to further evaluate the production of monatin in a single bioreactor at lower temperatures, as compared to 25° C. The three step pathway for the production of monatin (see reactions (6)-(8) above) was carried out at 3 L final volume in 5 L BioFlo 3000 (New Brunswick) vessels. Each bioreactor was configured for automated control of agitation, temperature and pH.

Solutions containing 130 mM D-tryptophan, 200 mM sodium pyruvate, 10 mM potassium phosphate (pH 7.8), 1 mM MgCl2, 0.01% (v/v) Tween 80 and 0.05 mM pyridoxal-5-phosphate (PLP) were prepared in the bioreactors. D-tryptophan, sodium pyruvate and potassium phosphate were added as solids, the remaining ingredients were added from degassed stock solutions.

The temperature of the mixture inside each reactor was maintained throughout the operation at the predetermined temperatures of 5, 13, 18, and 25 degrees Celsius. The pH was adjusted and then maintained at a pH of 7.8 by addition of potassium hydroxide to the bioreactor. A D-aminotransferase (see SEQ ID NO:1 and SEQ ID NO:2) and an aldolase (see SEQ ID NO:3 and SEQ ID NO:4) were added as clarified cell extracts to a final concentration of 0.2 and 0.02 g/L, respectively. The mixture inside the reactor was agitated at 250 rpm and maintained at the controlled temperature under a nitrogen headspace.

The progress of the reaction was followed by measuring pyruvate, indole-3-pyruvate, HMO, monatin precursor (MP) using the analytical method of Example 1. Tryptophan and monatin were measured using the analytical method of Example 2, and alanine and HMG were measured using the analytical method of Example 3.

The amount of monatin produced was measured as a function of reaction time, up to about 42 hours, and the results are shown in Table 2 below.

TABLE 2
Monatin formation (mM) over time at 5° C., 13° C., 18° C. and 25° C.

| 5 C. | | 13 C. | | 18 C. | | 25 C. | |
|---|---|---|---|---|---|---|---|
| | Monatin | | Monatin | | Monatin | | Monatin |
| hr | (mM) | Hr | (mM) | hr | (mM) | hr | (mM) |
| 0.0 | 0 | 0.0 | 0.4 | 0.0 | 0.8 | 0.0 | 0.5 |
| 0.2 | 0.2 | 0.2 | 0.6 | 0.2 | 1.1 | 0.2 | 1.1 |
| 0.5 | 0.5 | 0.5 | 1.1 | 0.5 | 1.7 | 0.5 | 2.1 |
| 0.8 | 0.9 | 0.8 | 1.5 | 0.8 | 2.5 | 0.8 | 2.6 |
| 1.0 | 1.1 | 1.0 | 1.9 | 1.0 | 3.5 | 1.0 | 3.7 |
| 2.0 | 2.3 | 2.0 | 3.7 | 2.0 | 5.7 | 2.0 | 6.5 |
| 3.0 | 3.6 | 3.0 | 5.8 | 3.0 | 7.5 | 3.0 | 8.3 |
| 4.0 | 4.9 | 4.0 | 7.5 | 4.0 | 9.1 | 4.0 | 10.2 |
| 15.4 | 15.5 | 16.0 | 19 | 16.0 | 18.4 | 15.4 | 16.4 |
| 23.0 | 18.4 | 22.0 | 21.3 | 22.0 | 20.2 | 22.0 | 17.9 |
| 27.9 | 20.7 | 28.0 | 22.4 | 29.4 | 19.9 | 28.0 | 18.1 |
| 39.4 | 24.8 | 39.3 | 25.23 | 40.0 | 26.25 | 43.1 | 19.6 |
| 42.0 | 26.8 | 41.1 | 27.3 | 42.3 | 26 | 43.0 | 18.7 |

The results in Table 2 are consistent with the results from Example 6 above, illustrating that the initial rate of monatin production is highest at 25° C., as compared to 5° C., 13° C. and 18° C. However, after approximately 22 hours, the amount of monatin produced is higher at 13° C. and 18° C. as compared to 25° C. At reaction times greater than about 28 hours, higher amounts of monatin are produced at 5° C., 13° C. and 18° C. as compared to 25° C. At 42 hours, a reaction temperature of 5° C. resulted in a 42% increase in monatin production, as compared to 25° C.; a reaction temperature of 13° C. resulted in a 46% increase; and a reaction temperature of 18° C. resulted in a 39% increase.

The concentration of monatin, as well as the various intermediates of the monatin producing reactions, are shown in Table 3 below, as measured at approximately 42 hours.

TABLE 3
Concentration of monatin and intermediates (mM) at 42 hours

| | 5° C. | 13° C. | 18° C. | 25° C. |
|---|---|---|---|---|
| Monatin | 26.8 | 27.3 | 25.9 | 18.7 |
| I3P | 36.0 | 39.0 | 49.6 | 44.6 |
| Alanine | 47.7 | 47.5 | 50.1 | 57.6 |
| Tryptophan | 39.7 | 38.9 | 41.0 | 40.3 |
| Pyruvate | 45.8 | 49.1 | 55.8 | 60.2 |
| HMO | 23.3 | 19.6 | 18.9 | 15.6 |
| HMG | 2.0 | 2.3 | 2.4 | 1.5 |
| MP | 22.9 | 19.7 | 17.8 | 15.8 |

### Example 8

Bench scale reactions at 7, 10, 13, 15, 18 and 20 degrees Celsius were carried out at 0.3 L final volume in 0.7 L INFORS agitated fermenters (Infors AG, Bottmingen, Switzerland) under a nitrogen headspace, and 200 rpm agitation. The bioreactors each contained a solution of 200 mM sodium pyruvate, 130 mM D-tryptophan, 5 mM sodium phosphate, 1 mM magnesium chloride, 0.01% Tween 80, and 0.02 mM pyridoxal-5-phosphate. The reaction mixture was prepared using degassed liquids and the pH was adjusted to and maintained at 7.8 with 1 M sodium hydroxide. An aldolase (see SEQ ID NO:3 and SEQ ID NO:4) was added as a clarified cell extract at 0.02 g/L and a D-aminotransferase (see SEQ ID NO:1 and SEQ ID NO:2) was added as a clarified cell extract at 0.20 g/L.

The progress of each reaction was followed by measuring indole-3-pyruvate (I3P), pyruvate, HMO, and monatin precursor (MP) using the analytical method of Example 1. Tryptophan, alanine, HMG and monatin were measured using the analytical method of Example 5. The analytical method of Example 4 was used to determine the stereoisomeric composition of the monatin in the mixture.

Table 4 below shows the rate of monatin formation up to 6 hours, at each of the temperatures, as well as the concentration of monatin formed at 72 hours and the percentage R,R monatin of the monatin formed.

TABLE 4
Monatin Formation at 7° C., 10° C., 13° C., 15° C., 18° C. and 20° C.

| | Monatin Formation | Monatin | |
|---|---|---|---|
| | Rate (mMoles/L*h) | (mM) @ | Final |
| | (0-6 hours) | 72 hours | % R,R |
| 7° C. | 0.89 | 31.3 | 99.1 |
| 10° C. | 1.08 | 31.1 | 98.8 |
| 13° C. | 1.63 | 31.2 | 98.6 |
| 15° C. | 1.84 | 29.7 | 98.0 |
| 18° C. | 1.91 | 26.3 | 97.7 |
| 20° C. | 1.83 | 26.3 | 97.3 |

The data in Table 4 show that the rate of monatin production between 0 and 6 hours generally increases as a function of an increase in the temperature of the reaction mixture. However, the concentration of monatin after 72 hours is higher at lower temperatures (i.e. 7° C., 10° C., and 13° C.) as compared to 18° C. and 20° C.

Table 5 below illustrates the concentration of monatin in the reaction mixture at the various temperatures at times between 0 and 72 hours.

TABLE 5
Monatin formation (mM) between 0 and 72 hours

| Sample time | | | | | | |
|---|---|---|---|---|---|---|
| (hrs) | 7° C. | 10° C. | 13° C. | 15° C. | 18° C. | 20° C. |
| 0 | 0.3 | 0.4 | 0.3 | 0.4 | 0.2 | 0.1 |
| 2 | 1.1 | 1.4 | 2.4 | 2.9 | 3.2 | 3.1 |
| 4 | 3.7 | 4.6 | 6.5 | 7.8 | 7.8 | 7.2 |
| 6 | 5.4 | 6.5 | 9.8 | 11.0 | 11.4 | 10.9 |
| 8 | 7.5 | 7.8 | 12.3 | 13.8 | 13.7 | 13.6 |
| 24 | 19.2 | 21.4 | 25.5 | 26.8 | 24.8 | 25.1 |
| 30 | 22.0 | 25.2 | 28.8 | 27.3 | 25.8 | 27.1 |
| 48 | 27.4 | 30.1 | 30.2 | 30.0 | 28.2 | 27.5 |
| 54 | 28.4 | 30.1 | 31.2 | 31.6 | 26.2 | 27.2 |
| 72 | 31.3 | 31.1 | 31.2 | 29.7 | 26.3 | 26.3 |

The results of Table 5 illustrates that up to 8 hours, monatin production at 7° C. and 10° C. is lower than monatin production at temperatures of 15° C. and higher. Similar to data represented in Table 4, at reaction times up to 8 hours, the amount of monatin produced increased with increasing temperature of the reaction mixture for temperatures between 7° C. and 15° C. However, between a reaction time of 24 and 30 hours, the difference in the amount of monatin produced at 7° C. and 10° C., as compared to temperatures of 18° C. and 20° C., is significantly reduced. At reaction times between 48 and 54 hours, the most monatin was produced at 10° C., 13° C. and 15° C. After 72 hours, the mixture at 7° C. produced an amount of monatin comparable to the mixtures at 10° C., 13° C. and 15° C., all of which were significantly higher than the concentration of monatin produced in the reactions at 18° C. and 20° C.

It is recognized that various modifications to the described invention may be made without departing from the spirit and scope of the disclosure. It is recognized that while the invention has been described in conjunction with the detailed description thereof, the foregoing description is intended to illustrate and not limit the scope of the invention. Accordingly, other embodiments are within the scope of the following claims.

What is claimed is:

1. A method of making monatin in a multi-step equilibrium pathway, the method comprising:
adding tryptophan and pyruvate to a reactor to form a mixture comprising monatin and a plurality of intermediates via a multi-step equilibrium pathway;
adding at least one enzyme to the reactor to facilitate at least one reaction in the multi-step equilibrium pathway; and
operating the reactor under conditions such that a temperature of the mixture in the reactor is between about 5 degrees Celsius and about 23 degrees Celsius.

2. The method of claim 1 wherein the temperature of the mixture in the reactor is between about 10 degrees Celsius and about 18 degrees Celsius.

3. The method of claim 1 wherein the temperature of the mixture in the reactor is between about 12 degrees Celsius and about 16 degrees Celsius.

4. The method of claim 1 wherein a pH of the mixture in the reactor is between about 7 and about 9.

5. The method of claim 1 wherein the multi-step equilibrium pathway includes a conversion of tryptophan to indole-3-pyruvate, indole-3-pyruvate to 2-hydroxy 2-(indol -3-ylmethyl)-4-keto glutaric (MP), and MP to monatin.

6. The method of claim 1 wherein the at least one enzyme is selected from the group consisting of an aminotransferase, a racemase, and an aldolase.

7. The method of claim 1 wherein tryptophan is D-tryptophan and the at least one enzyme includes a D-aminotransferase.

8. The method of claim 1 wherein the monatin in the mixture is a stereoisomerically-enriched R,R monatin.

9. A method of producing a stereoisomerically-enriched R,R monatin comprising:
adding D-tryptophan and pyruvate to a reactor to produce a mixture of stereoisomerically-enriched R,R monatin and intermediates, wherein the tryptophan is converted to indole-3-pyruvate, indole-3-pyruvate to 2-hydroxy 2-(indol-3-ylmethyl)-4-keto glutaric (R-MP), and R-MP to monatin;
adding a D-aminotransferase and an R-specific aldolase to the reactor, wherein the D-aminotransferase catalyzes at least one of the conversion of D-tryptophan to indole-3-pyruvate and R-MP to R,R-monatin, and the R-specific aldolase catalyzes the conversion of indole-3-pyruvate to R-MP; and
adding at least one additive to the reactor to stabilize at least one of the intermediates, the D-aminotransferase and the R-specific aldolase; and
maintaining a temperature of the mixture between about 5 degrees Celsius and about 23 degrees Celsius.

10. The method of claim 9 wherein the temperature is maintained between about 10 degrees Celsius and about 18 degrees Celsius.

11. The method of claim 9 wherein the temperature is maintained between about 12 degrees Celsius and about 16 degrees Celsius.

12. The method of claim 9 wherein the temperature of the mixture is maintained at about 15 degrees Celsius.

13. The method of claim 9 wherein the additive is an enzymatic cofactor.

14. The method of claim 13 wherein the additive includes at least one of potassium phosphate, sodium phosphate, magnesium chloride, pyridoxal phosphate, and a surfactant.

15. The method of claim 9 wherein adding D-tryptophan to the reactor includes adding an amount of D-tryptophan such that a concentration of D-tryptophan in the mixture is above the solubility limit of D-tryptophan in the mixture.

16. The method of claim 9 further comprising:
maintaining a pH of the mixture between about 7 and about 9.

17. The method of claim 16 wherein maintaining the pH of the mixture includes adding a hydroxide to the reactor.

18. The method of claim 17 wherein the hydroxide includes at least one of sodium hydroxide and potassium hydroxide.

19. The method of claim 9 further comprising:
removing the mixture from the reactor after at least about 8 hours.

20. The method of claim 9 further comprising:
removing the mixture from the reactor after at least about 24 hours.

21. A method of producing monatin comprising:
adding tryptophan and pyruvate to a reactor to produce a mixture of monatin and intermediates via a multi-step equilibrium pathway in which tryptophan is converted to indole-3-pyruvate, indole-3-pyruvate is converted to 2-hydroxy 2-(indol-3-ylmethyl)-4-keto glutaric (MP), and MP is converted to monatin;
adding an aminotransferase to the reactor to catalyze the conversion of tryptophan to indole-3-pyruvate and the conversion of MP to monatin;
adding an aldolase to the reactor to catalyze the conversion of indole-3-pyruvate to MP;
maintaining a temperature of the mixture at a first temperature for a predetermined time, wherein the first temperature is less than or equal to about 25 degrees Celsius; and
maintaining a temperature of the mixture at a second temperature after the predetermined time, wherein the second temperature is less than the first temperature.

22. The method of claim 21 wherein the second temperature is less than or equal to about 15 degrees Celsius.

23. The method of claim 21 wherein the second temperature is between about 10 and about 18 degrees Celsius.

24. The method of claim 21 wherein the predetermined time is less than or equal to about 8 hours.
